(19) United States
(12) Patent Application Publication
(10) Pub. No.: US 2011/0038755 A1
(43) Pub. Date: Feb. 17, 2011

(54) CONTAINERS COMPRISING PEELABLE SEALS

(75) Inventors: Giampiero Pesci, Tirano (IT); Patrick R. Balteau, Evelette (BE); Jean-Pierre Hartman, Rhode-St-Genese (BE); Noel G. Smith, County Mayo (IE); Fabio Giuseppe Casartelli, Sondrio (IT)

Correspondence Address: K&L Gates LLP, P.O. Box 1135, Chicago, IL, 60690-1135, US

(73) Assignees: BAXTER INTERNATIONAL INC., Deerfield, IL (US); BAXTER HEALTHCARE S.A., Zurich (CH)

(21) Appl. No.: 12/539,948

(22) Filed: Aug. 12, 2009

(51) Int. Cl. A61L 2/04; B65D 25/04

(52) U.S. Cl. 422/26; 220/529

(57) ABSTRACT

Multi-chambered containers having peelable seals and methods of sterilizing the same are provided. In an embodiment, the present disclosure provides a flexible fluid container comprising a first chamber and a second chamber. The second chamber is attached to a tube for providing fluid communication from the second chamber to the exterior of the container. The first chamber and the second chamber are separated by a first peelable seal having a channel so constructed and arranged to allow a controlled quantity of fluid to pass therethrough.

### BACKGROUND

The present disclosure is generally directed to packaging products. More specifically, the present disclosure is directed to containers having peelable seals and methods of sterilizing such containers.

Containers having sub-chambers are widely used to separately store two or more components. The components can be mixed inside the container and administered to a patient through a tube attached to the container. The components can be in a powder or liquid form and are typically mixed together to form a solution, for example a therapeutic solution. Such therapeutic solutions can include intravenous base solutions, parenteral and enteral nutritional solutions, drug solutions, dialysis solutions, pharmacological agents including gene therapy and chemotherapy agents, and many other liquids that may be administered to a patient.

The chambers and solutions of the multi-chambered containers are often separated by a frangible seal. Peelable seals are among the frangible seals used that permit the seal to be separated by pulling on opposite sides of the container.

After the chambers of the container are filled with solutions, the container is typically steam sterilized at a temperature of 121° C. During the steam sterilization, pressure typically builds on a side of the peelable seal next the access port tube, which can deform the tube as a result of a partial vacuum created during the cooling phase of the steam sterilization cycle. The deformed tube can cause problems when subsequently used for administering the solutions of the container.

### SUMMARY

The present disclosure is directed to containers having peelable seals and methods of sterilizing the containers. In a general embodiment, the present disclosure provides a flexible liquid container comprising a first chamber and a second chamber. The second chamber is attached to a tube for providing fluid communication from the second chamber to the exterior of the container. The first chamber and the second chamber are separated by a first peelable seal having a channel so constructed and arranged to allow a controlled quantity of liquid to pass therethrough. The channel allows for sufficiently equalized pressure on both sides of the second peelable seal at any time during a sterilization cycle (e.g. using steam/heat) that the container may undergo.

In an embodiment, the first peelable seal surrounds an opening of the tube attached to the container. The tube can be sealed until the time to access the contents of the container.

In an embodiment, the channel has a width greater than about 5 mm. The channel can also have a larger width such as, for example, a width greater than 6 mm or 7 mm.

In an embodiment, the container is made from a film comprising one or more materials such as polyolefins, polyamides, polyesters, polybutadiene, styrene and hydrocarbon copolymers, polyimides, polyester-polyethers, polyamide-polyethers, or a combination thereof. More specifically, the films can comprise one or more materials such as polyethylene homopolymers, ethylene α-olefin copolymers, polyethylene copolymers, polypropylene homopolymers, polypropylene copolymers, styrene and hydrocarbon random copolymers, styrene and hydrocarbon block copolymers, or a combination thereof.

In an embodiment, the first chamber is divided into a plurality of subchambers by one or more openable seals. One or more of these openable seals can be a second peelable seal.

In an embodiment, the first peelable seal and the second peelable seal can be activated by a force ranging from about 3 N/15 mm to about 30 N/15 mm. In another embodiment, the first peelable seal has a first activating force and the second peelable seal has a second activating force. The second peelable seal activating force is greater than the first peelable seal activating force. The difference between the first peelable seal activating force and the second peelable seal activating force can be greater than about 1 N/15 mm and less than about 10 N/15 mm.

In an embodiment, the first peelable seal is openable in response to liquid pressure applied to the first peelable seal. Similarly, the second peelable seal can be openable in response to liquid pressure applied to the second peelable seal.

In an embodiment, at least a portion of the second peelable seal comprises a shape such as semicircular, rectangular, trapezoidal, polygonal, or combinations thereof. In another embodiment, at least a portion of the channel comprises a shape such as straight, rectangular, trapezoidal, polygonal, or combinations thereof.

In an alternative embodiment, the present disclosure provides a method of sterilizing a container. The method comprises providing a container comprising a first chamber comprising a liquid and a second chamber connected to a tube. The first chamber and the second chamber are separated by a first peelable seal having a channel so constructed and arranged to allow a controlled quantity of the liquid to pass therethrough. The method further comprises sterilizing the container and allowing the liquid from the first chamber to flow through the channel into the second chamber to equalize the pressure in the first chamber and the second chamber. The sterilization of the container can be performed by heat sterilization.

An advantage of the present disclosure is to provide an improved container having a tube that does not deform during sterilization.

Another advantage of the present disclosure is to provide an improved multi-chambered container having peelable seals.

Yet another advantage of the present disclosure is to provide a method of sterilizing a multi-chambered container.

Additional features and advantages are described herein, and will be apparent from the following Detailed Description and the figures.

### BRIEF DESCRIPTION OF THE FIGURES

FIG. 1 illustrates a front perspective view of the container comprising a peelable seal with a channel in a first embodiment of the present disclosure.

FIG. 2 illustrates a front perspective view of the multi-chambered container comprising a peelable seal with a channel in a second embodiment of the present disclosure.

### DETAILED DESCRIPTION

The present disclosure relates to containers having one or more peelable seals and methods of sterilizing the containers. The containers can have multiple chambers used to deliver reconstituted solutions starting from concentrates that are separately stored in each chamber. A unique peelable seal having a thin channel can be used to isolate a tube attached to the container from an adjacent solution. Liquid or another incompressible fluid contained in the container can percolate through the channel and equilibrate the pressure in the chambers on either side of the peelable seal. As used herein, the term “liquid” means substances such as water, oil, alcohol, and the like, that are neither solids nor gases. The term “incompressible fluid” includes liquids as well as flowable solids such as powders.

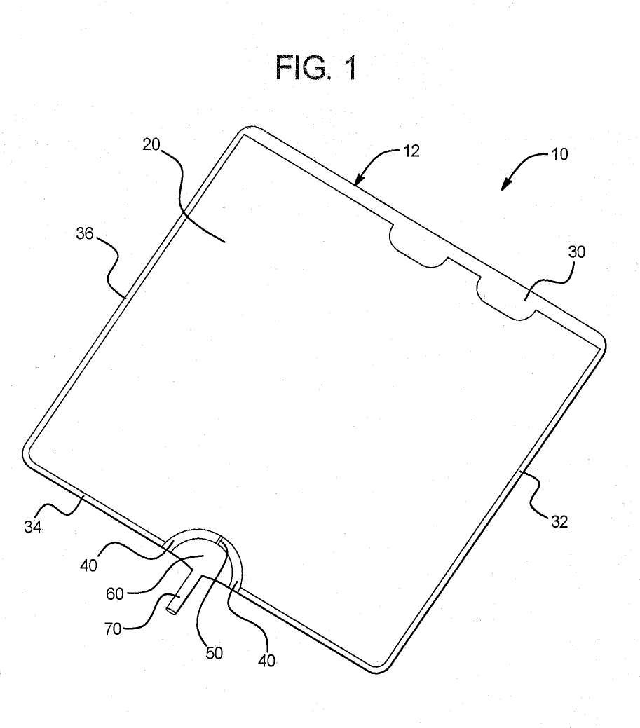

In a general embodiment shown in FIG. 1, the present disclosure provides a flexible liquid container 10 comprising a body 12 defined by a film. The flexible liquid container 10 comprises a chamber 20. The container 10 may be made from two sheets of the film that are, for example, sealed (for example, heat sealed) along their edges (30, 32, 34, and 36) to form permanent seals. In the illustrated embodiment, two sheets of film are used. The sheets are sealed about the periphery of the container 10 at edges 30, 32, 34, and 36. A peelable seal 40 is provided between the sheets of film to form the chamber 20 and another chamber 60.

In the illustrated embodiment, any portion of the container 10 can be made from one or more suitable polymer materials. Suitable polymer materials include polyethylene homopolymers, ethylene α-olefin copolymers, polyethylene copolymers, polypropylene homopolymers, polypropylene copolymers, styrene and hydrocarbon random copolymers, styrene and hydrocarbon block copolymers, or combinations thereof. More examples of suitable polymer materials are described below.

The peelable seal 40 comprises a channel 50 that is constructed and arranged to allow a controlled quantity of liquid to pass through the peelable seal 40 from chamber 20 to chamber 60 (and vice versa). The peelable seal 40 can be positioned to surround an opening of a tube 70 attached to chamber 60. It should be appreciated that the channel 50 exists in the peelable seal 40 before the peelable seal 40 is activated, opened, or unpeeled.

The channel 50 has a sufficient diameter to allow for a sufficiently equalized pressure on both sides of the peelable seal at any time during a sterilization cycle (e.g. using steam/heat) to prevent deformation of the tube 70. For example, during the steam/heat sterilization of the container 10, the amount of liquid that will have migrated into chamber 60 is enough to equilibrate the pressure on either side of the peelable seal 40, thereby avoiding the deformation of the tube 70 that would otherwise deform itself (or collapse) as a result of a partial vacuum created during the cooling phase of the steam sterilization cycle if channel 50 were not present or were too narrow.

The channel 50 can be sized so that the quantity or rate of liquid flowing through the channel 50 is less than the quantity or rate of liquid that can flow through the tube 70. This can prevent or mitigate liquid flow from the chamber 20 through the tube 70 when the peelable seal 40 is not opened. In an embodiment, the maximum flow rate of the liquid that can pass through the channel 50 is less than 20% of the maximum flow rate of the liquid that can pass through the tube 70. In alternative embodiments, the maximum flow rate of the liquid that can pass through the channel 50 is less than 19%, 18%, 17%, 16%, 15%, 14%, 13%, 12%, 11%, 10%, 9%, 8%, 7%, 6%, 5%, 4%, 3%, 2%, 1%, and the like, of the maximum flow rate of the liquid that can pass through the tube 70.

In an embodiment, the channel has a width greater than 5 mm. The channel can also have a larger width such as, for example, a width greater than 6 mm, greater than 7 mm, greater than 8 mm, greater than 9 mm, greater than 10 mm, and the like.

The peelable seal and the channel can comprise any suitable shape. In an embodiment, at least a portion of the peelable seal comprises a shape such as semicircular, rectangular, trapezoidal, polygonal, or combinations thereof. In another embodiment, at least a portion of the channel comprises a shape such as straight, rectangular, trapezoidal, polygonal, or combinations thereof.

The tube 70 allows liquid to be added to or removed from the container. The tube 70 can also include a membrane (not shown) that seals shut the tube and can be pierced by, for example, a cannula or spike of an administration set. The tube 70 can be sealed until the time to access the contents of the container 10.

The solutions stored in the container can provide a therapeutic solution. Such solutions can include intravenous solutions, nutritional solutions, drug solutions, enteral solutions, parenteral solutions, dialysis solutions, and many other liquids that may be administered to a patient.

The container 10 and the peelable seal 40 can be constructed from films able to make peal seal layers in accordance with embodiments of the present disclosure. The peelable seal layer films can allow both peelable and permanent seals to be created. Thus, the permanent side seals 30, 32, 34, and 36 as well as the peelable seal 40 can be created from the same layer of film.

The peelable seal can be openable by pulling on the sides of the container near the peelable seal. Alternatively, the peelable seal can be openable in response to liquid pressure applied to the peelable seal.

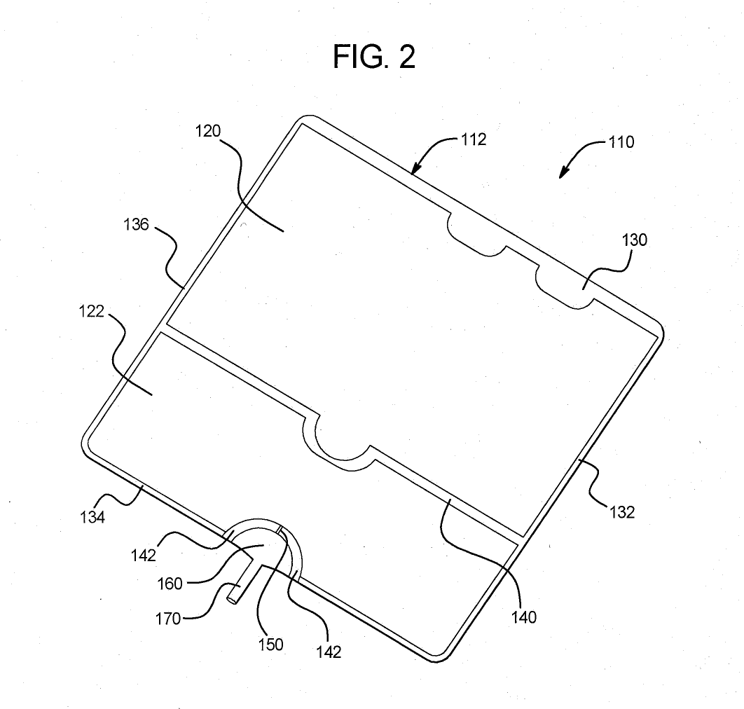

In another embodiment shown in FIG. 2, the present disclosure provides a multi-chambered container 110 comprising a body 112 defined by a film. The multi-chambered container 110 comprises two chambers 120 and 122. It should be appreciated that in alternative embodiments more than two chambers can be provided in the container. The chambers 120 and 122 are designed for the separate storage of substances and/or solutions. Any portion of the container 110 can be made from one or more suitable polymer materials.

The container 110 may be made from two sheets of the film that are, for example, heat sealed along their edges (130, 132, 134, and 136) to form permanent seals. In the illustrated embodiment, two sheets of film are used. The sheets are sealed about the periphery of the container 110 at edges 130, 132, 134, and 136. A first peelable seal 140 is provided between the sheets of film to form the chambers 120 and 122. Of course, if additional chambers are desired, additional peelable seals can be provided.

A second peelable seal 142 comprising a channel 150 is also provided between the sheets of film to form an additional chamber 160. In an embodiment, the channel 150 is constructed and arranged to allow a controlled quantity of liquid to pass through the second peelable seal 142 from chamber 122 to chamber 160 in a manner previously discussed. It should be appreciated that the channel 150 exists in the second peelable seal 142 prior to the second peelable seal 150 being activated, opened or unpeeled. The first and second peelable seals and the channel can comprise any suitable shape.

As illustrated in FIG. 2, the container 110 can further comprise a tube 170 attached to an end of the container having the third chamber 160. The second peelable seal 142 can surround an opening of the tube 170. The tube 170 provides communication with the interior of chamber 160. The tube 170 allows liquid to be added to or removed from the multi-chambered container, for example, after the contents of chambers 120 and 122 have been mixed. The tube 170 can also include a membrane (not shown) that seals shut the tube and can be pierced by, for example, a cannula or spike of an administration set. The tube 170 can be sealed until the time to access the contents of the container 110.

The first peelable seal 140 is used to separate chamber 120 and chamber 122 where two solutions (e.g. in the form of concentrates) can be stored during the manufacturing process. The second peelable seal 142 can be used to isolate the container tube 170 from the content of chamber 122. The solutions stored in the two chambers, when mixed together immediately prior usage, can provide a reconstituted therapeutic solution. Such solutions can include intravenous solutions, nutritional solutions, drug solutions, enteral solutions, parenteral solutions, dialysis solutions, and many other liquids that may be administered to a patient.

In an embodiment, the first peelable seal 140 and the second peelable seal 142 can be activated by a force ranging from about 3 N/15 mm to about 30 N/15 mm. The force can be applied, for example, by pulling the sides of the container away from each other. In another embodiment, the first peelable seal 140 has a first activating force (e.g. amount of force necessary to open the seal) and the second peelable seal 142 has a second activating force. The second peelable seal activating force is greater than the first peelable seal activating force. The difference between the first peelable seal activating force and the second peelable seal activating force can be greater than about 1 N/1 5 mm and less than about 5 N/15 mm.

In addition to pulling on the sides of the container near the peelable seals, the first peelable seal 140 can be openable in response to liquid pressure applied to the first peelable seal 140. Similarly, the second peelable seal 142 can be openable in response to liquid pressure applied to the second peelable seal 142.

In another embodiment, the present disclosure provides a method of sterilizing a container. The method comprises providing a container comprising a first chamber comprising a first liquid, a second chamber comprising a second liquid, and a third chamber connected to a tube. The first chamber and second chamber are separated by a first peelable seal. The second chamber and the third chamber are separated by a second peelable seal having a channel constructed and arranged to allow liquid to pass through.

The method further comprises sterilizing the container. During this time, the second liquid from the second chamber can flow through the channel into the third chamber. This sufficiently equalizes the pressure in the second chamber and the third chamber and avoids deformation of the tube. The sterilization of the container can be performed, for example, by heat sterilization.

The multi-chambered container can be operated in any suitable manner. For example, the container can be squeezed at the end opposing the tube. Squeezing this end will open the first peelable seal allowing the solutions of the first and second chamber to mix. Continuous squeezing will then open the second seal allowing the mixed contents to flow into the tube where it can be administered to a recipient.

The containers in alternative embodiments of the present disclosure can be fabricated using standard sealing techniques. For example, the container can be typically formed by placing one or more polymeric film sheets forming the first sidewall and second sidewall of the container body in registration by their peripheral portions and sealing their periphery to form a liquid tight pouch having an outer permanent seal. The polymeric film sheets can be sealed, for example, by heat sealing, radio frequency sealing, thermal transfer welding, adhesive sealing, solvent bonding, and ultrasonic or laser welding.

The peelable seals can be formed prior to, during or after forming the permanent seal and can be made, for example, by using heat conduction sealing techniques. The welding die for the peelable seals may have different temperatures and shapes along its length to achieve the desired peelable seal configurations.

Blown extrusion is another method that may be used to make the pouch. Blown extrusion is a process that provides a moving tube of extrudate exiting an extrusion die. Air under pressure inflates the tube. Longitudinal ends of the tube are sealed to form the pouch. Blown extrusion only requires seals along two peripheral surfaces, where the single or multiple sheet registration method typically requires seals along three (if the film is folded) or four peripheral surfaces to form the pouch.

### The Sidewall Materials and Layer Structures

The container 10 can be made principally of flexible polymeric materials, although the container could include non-polymeric materials such as metal foils without departing from the disclosure. Numerous polymeric films have been developed for use in containers. Suitable films may be of a monolayer structure or a multiple layer structure. The monolayer structure can be made from a single polymer, or from a polymer blend. The multiple layer structures can include layers such as a solution contact layer, a scratch resistant layer, a barrier layer for preventing permeation of oxygen or water vapor, tie layers, or other layers. It is also contemplated to use more than one web of film for one or both sidewalls. Selection of the appropriate film depends on the solution or solutions to be contained within the container. Appropriate polymeric materials are generally selected from homopolymers and copolymers of polyolefins, polyamides, polyesters, polybutadiene, styrene and hydrocarbon copolymers, polyimides, polyester-polyethers, polyamide-polyethers to name a few.

The seal layer for a multi-chambered container should display bi-modal behavior. What is meant by bi-modal behavior is that the material is capable of forming a permanent (cohesive) seal under one set of sealing or manufacturing conditions and a peelable (adhesive) seal at a second set of sealing or manufacturing conditions. The seal layer can be a homophase polymer, or a matrix-phase polymer system. Suitable homophase polymers include polyolefins and more preferably polypropylene and most preferably a propylene and ethylene copolymer as described in EP 0875231, which is incorporated herein by reference.

It is also possible to have a seal layer having container walls of differing materials that are not compatible with one another. U.S. patent application Ser. No. 10/351,004, which is incorporated herein by reference, discloses that containers made from such incompatible material, in some instances, may not readily form permanent seals. This problem can be overcome by wrapping a section of one sidewall over an outside surface of the opposite sidewall and joined thereto. This method of sealing is disclosed in U.S. Pat. No. 6,024,220 which is incorporated herein by reference and made a part hereof.

Suitable matrix-phase polymer systems will have at least two components. The two components can be blended together or can be produced in a two-stage reactor process. Typically, the two components will have different melting point or glass transition temperatures. In the case where one of the components is amorphous, its glass transition temperature will be lower than the melting point of the other components. One illustrative example of a suitable matrix-phase polymer system includes a component of a homopolymer or copolymer of a polyolefin and a second component of a styrene and hydrocarbon copolymer. Another suitable matrix-phase system includes blends of polyolefins such as polypropylene with polyethylene, or polypropylene with a high isotactic index (crystalline) with polypropylene with a lower isotactic index (amorphous), or a polypropylene homopolymer with a propylene and α-olefin copolymer.

Suitable polyolefins include homopolymers and copolymers obtained by polymerizing alpha-olefins containing from 2 to 20 carbon atoms, and more preferably from 2 to 10 carbons. Therefore, suitable polyolefins include polymers and copolymers of propylene, ethylene, butene-1, pentene-1, 4-methyl-1-pentene, hexene-1, heptene-1, octene-1, nonene-1 and decene-1. Most preferably the polyolefin is a homopolymer or copolymer of propylene or a homopolymer or copolymer of polyethylene.

Suitable homopolymers of polypropylene can have a stereochemistry of amorphous, isotactic, syndiotactic, atactic, hemiisotactic or stereoblock. In one embodiment, the homopolymer of polypropylene is obtained using a single site catalyst.

Suitable copolymers of propylene are obtained by polymerizing a propylene monomer with an α-olefin having from 2 to 20 carbons. In a more preferred form of the disclosure, the propylene is copolymerized with ethylene in an amount by weight from about 1% to about 20%, more preferably from about 1% to about 10% and most preferably from 2% to about 5% by weight of the copolymer. The propylene and ethylene copolymers may be random or block copolymers. The propylene copolymer may also be obtained using a single site catalyst.

It is also possible to use a blend of polypropylene and α-olefin copolymers wherein the propylene copolymers can vary by the number of carbons in the α-olefin. For example, the present disclosure contemplates blends of propylene and α-olefin copolymers wherein one copolymer has a 2 carbon α-olefin and another copolymer has a 4 carbon α-olefin. It is also possible to use any combination of α-olefins from 2 to 20 carbons and most preferably from 2 to 8 carbons. Accordingly, the present disclosure contemplates blends of propylene and α-olefin copolymers wherein the first and second α-olefins have the following combination of carbon numbers: 2 and 6, 2 and 8, 4 and 6, 4 and 8. It is also contemplated using more than 2 polypropylene and α-olefin copolymers in the blend. Suitable polymers can be obtained using a catalloy procedure. Suitable homopolymers of ethylene include those having a density of greater than 0.915 g/cc and includes low density polyethylene (LDPE), medium density polyethylene (MDPE) and high density polyethylene (HDPE).

Suitable copolymers of ethylene are obtained by polymerizing ethylene monomers with an α-olefin having from 3 to 20 carbons, more preferably 3-10 carbons and most preferably from 4 to 8 carbons. It is also desirable for the copolymers of ethylene to have a density as measured by ASTM D-792 of less than about 0.915 g/cc and more preferably less than about 0.910 g/cc and even more preferably less than about 0.900 g/cc. Such polymers are oftentimes referred to as VLDPE (very low density polyethylene) or ULDPE (ultra low density polyethylene). Preferably the ethylene α-olefin copolymers are produced using a single site catalyst and even more preferably a metallocene catalyst system. Single site catalysts are believed to have a single, sterically and electronically equivalent catalyst position as opposed to the Ziegler-Natta type catalysts which are known to have a mixture of catalyst sites. Such single-site catalyzed ethylene α-olefins are sold by Dow under the trade name AFFINITY, DuPont Dow under the trademark ENGAGE®, Eastman Kodak under the trade name MXSTEN, and by Exxon under the trade name EXACT. These copolymers shall sometimes be referred to herein as m-ULDPE.

Suitable copolymers of ethylene also include ethylene and lower alkyl acrylate copolymers, ethylene and lower alkyl substituted alkyl acrylate copolymers and ethylene vinyl acetate copolymers having a vinyl acetate content of from about 8% to about 40% by weight of the copolymer. The term “lower alkyl acrylates” refers to comonomers having the formula set forth in Diagram 1:

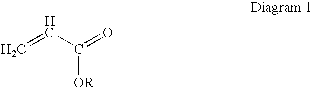

The R group refers to alkyls having from 1 to 17 carbons. Thus, the term “lower alkyl acrylates” includes but is not limited to methyl acrylate, ethyl acrylate, butyl acrylate and the like.

The term “alkyl substituted alkyl acrylates” refers to comonomers having the formula set forth in Diagram 2:

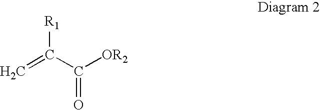

R₁ and R₂ are alkyls having 1-17 carbons and can have the same number of carbons or have a different number of carbons. Thus, the term “alkyl substituted alkyl acrylates” includes but is not limited to methyl methacrylate, ethyl methacrylate, methyl ethacrylate, ethyl ethacrylate, butyl methacrylate, butyl ethacrylate and the like.

Suitable polybutadienes include the 1,2- and 1,4-addition products of 1,3-butadiene (these shall collectively be referred to as polybutadienes). In a more preferred form of the disclosure, the polymer is a 1,2-addition product of 1,3 butadiene (these shall be referred to as 1,2 polybutadienes). In an even more preferred form of the disclosure, the polymer of interest is a syndiotactic 1,2-polybutadiene and even more preferably a low crystallinity, syndiotactic 1,2 polybutadiene. In a preferred form of the disclosure, the low crystallinity, syndiotactic 1,2 polybutadiene will have a crystallinity less than 50%, more preferably less than about 45%, even more preferably less than about 40%, even more preferably the crystallinity will be from about 13% to about 40%, and most preferably from about 15% to about 30%. In a preferred form of the disclosure, the low crystallinity, syndiotactic 1,2 polybutadiene will have a melting point temperature measured in accordance with ASTM D 3418 from about 70° C. to about 120° C. Suitable resins include those sold by JSR (Japan Synthetic Rubber) under the grade designations: JSR RB 810, JSR RB 820, and JSR RB 830.

Suitable polyesters include polycondensation products of di-or polycarboxylic acids and di or poly hydroxy alcohols or alkylene oxides. In a preferred form of the disclosure, the polyester is a polyester ether. Suitable polyester ethers are obtained from reacting 1,4 cyclohexane dimethanol, 1,4 cyclohexane dicarboxylic acid and polytetramethylene glycol ether and shall be referred to generally as PCCE. Suitable PCCE's are sold by Eastman under the trade name ECDEL. Suitable polyesters further include polyester elastomers which are block copolymers of a hard crystalline segment of polybutylene terephthalate and a second segment of a soft (amorphous) polyether glycol. Such polyester elastomers are sold by DuPont Chemical Company under the trade name HYTREL®.

Suitable polyamides include those that result from a ring-opening reaction of lactams having from 4-12 carbons. This group of polyamides therefore includes nylon 6, nylon 10 and nylon 12. Acceptable polyamides also include aliphatic polyamides resulting from the condensation reaction of di-amines having a carbon number within a range of 2-13, aliphatic polyamides resulting from a condensation reaction of di-acids having a carbon number within a range of 2-13, polyamides resulting from the condensation reaction of dimer fatty acids, and amide containing copolymers. Thus, suitable aliphatic polyamides include, for example, nylon 6,6, nylon 6,10 and dimer fatty acid polyamides.,

Suitable styrene and hydrocarbon copolymers include styrene and the various substituted styrenes including alkyl substituted styrene and halogen substituted styrene. The alkyl group can contain from 1 to about 6 carbon atoms. Specific examples of substituted styrenes include alpha-methylstyrene, beta-methylstyrene, vinyltoluene, 3-methylstyrene, 4-methlylstyrene, 4-isopropylstyrene, 2,4-dimethylstyrene, o-chlorostyrene, p-chlorostyrene, o-bromostyrene, 2-chloro-4-methylstyrene, etc. Styrene is the most preferred.

The hydrocarbon portion of the styrene and hydrocarbon copolymer includes conjugated dienes. Conjugated dienes which may be utilized are those containing from 4 to about 10 carbon atoms and more generally, from 4 to 6 carbon atoms. Examples include 1,3-butadiene, 2-methyl-1,3-butadiene (isoprene), 2,3-dimethyl-1,3-butadiene, chloroprene, 1,3-pentadiene, 1,3-hexadiene, etc. Mixtures of these conjugated dienes also may be used such as mixtures of butadiene and isoprene. The preferred conjugated dienes are isoprene and 1,3-butadiene.

The styrene and hydrocarbon copolymers can be block copolymers including di-block, tri-block, multi-block, star block and mixtures of the same. Specific examples of di-block copolymers include styrene-butadiene, styrene-isoprene, and the hydrogenated derivatives thereof. Examples of tri-block polymers include styrene-butadiene-styrene, styrene-isoprene-styrene, alpha-methylstyrene-butadiene-alpha-methylstyrene, and alpha-methylstyrene-isoprene-alpha-methylstyrene and hydrogenated derivatives thereof.

The selective hydrogenation of the above block copolymers may be carried out by a variety of well known processes including hydrogenation in the presence of such catalysts as Raney nickel, noble metals such as platinum, palladium, etc., and soluble transition metal catalysts. Suitable hydrogenation processes which can be used are those wherein the diene-containing polymer or copolymer is dissolved in an inert hydrocarbon diluent such as cyclohexane and hydrogenated by reaction with hydrogen in the presence of a soluble hydrogenation catalyst. Such procedures are described in U.S. Pat. Nos. 3,113,986 and 4,226,952, the disclosures of which are incorporated herein by reference and made a part hereof.

Particularly useful hydrogenated block copolymers are the hydrogenated block copolymers of styrene-isoprene-styrene, such as a styrene-(ethylene/propylene)-styrene block polymer. When a polystyrene-polybutadiene-polystyrene block copolymer is hydrogenated, the resulting product resembles a regular copolymer block of ethylene and 1 -butene (EB). As noted above, when the conjugated diene employed is isoprene, the resulting hydrogenated product resembles a regular copolymer block of ethylene and propylene (EP). One example of a commercially available selectively hydrogenated copolymer is KRATON® G-1652 which is a hydrogenated SBS tri-block comprising 30% styrene end blocks and a mid-block equivalent is a copolymer of ethylene and 1-butene (EB). This hydrogenated block copolymer is often referred to as SEBS. Other suitable SEBS or SIS copolymers are sold by Kuraray under the tradename SEPTON® and HYBRAR®. It may also be desirable to use graft modified styrene and hydrocarbon block copolymers by grafting an alpha, beta-unsaturated monocarboxylic or dicarboxylic acid reagent onto the selectively hydrogenated block copolymers described above.

The block copolymers of the conjugated diene and the vinyl aromatic compound are grafted with an alpha, beta-unsaturated monocarboxylic or dicarboxylic acid reagent. The carboxylic acid reagents include carboxylic acids per se and their functional derivatives such as anhydrides, imides, metal salts, esters, etc., which are capable of being grafted onto the selectively hydrogenated block copolymer. The grafted polymer will usually contain from about 0.1 to about 20%, and preferably from about 0.1 to about 10% by weight based on the total weight of the block copolymer and the carboxylic acid reagent of the grafted carboxylic acid. Specific examples of useful monobasic carboxylic acids include acrylic acid, methacrylic acid, cinnamic acid, crotonic acid, acrylic anhydride, sodium acrylate, calcium acrylate and magnesium acrylate, etc. Examples of dicarboxylic acids and useful derivatives thereof include maleic acid, maleic anhydride, fumaric acid, mesaconic acid, itaconic acid, citraconic acid, itaconic anhydride, citraconic anhydride, monomethyl maleate, monosodium maleate, etc. The styrene and hydrocarbon block copolymer can be modified with an oil such as the oil modified SEBS sold by-the Shell Chemical Company under the product designation KRATON G2705.

The films used in the containers of the present disclosure can be a multiple layer film having a seal layer, an intermediate layer, and an external layer. Tie layers may be employed to attach the seal layer to the intermediate layer and to attach the intermediate layer to the external layer. In a preferred form of the disclosure the seal layer is a blend of polypropylene, an ethylene α-olefin copolymer and a styrene and hydrocarbon copolymer. In a more preferred form of the disclosure, the polypropylene is a polypropylene ethylene copolymer, the ethylene α-olefin copolymer is a LLDPE having a density of less than 0.915 g/cc and the styrene and hydrocarbon copolymer is a block copolymer and preferably a tri-block copolymer of styrene-ethylene-butylene-styrene or a blend of an SEBS tri-block with an SEBS di-block as a minor component. The relative proportions of the components are preferably from about 55% to 75% of the PP by weight, from 5% to 20% by weight of the LLDPE, and from 10% to 20% by weight of the SEBS. The ternary blend of the seal layer is capable of forming a permanent seal and a peelable seal at a temperature of from about 123 to 128° C. A permanent seal is achieved at sealing temperatures above 160° C.

The intermediate layer may be selected from any of the polyamides set forth herein and most preferably is a blend of from about 85 to 98% polyamide 6 and from 2 to 15% polyamide 616T. The external layer can be selected from polypropylene polymer. For example, the external layer can be a propylene ethylene copolymer with an ethylene content of less than 6% by weight of the copolymer.

The details of the films are more fully set out in U.S. patent application Ser. No. 09/439,826, filed Nov. 12, 1999, which is incorporated in its entirety herein by reference and made a part hereof.

Another suitable film can comprise three layers: an external layer, intermediate layer, and seal layer. The external layer can be a reactor made polypropylene composition having a first component and a second component. The first component can be a polypropylene homopolymer and can be present in an amount by weight of the composition of 40%. The second component can be an ethylene-propylene rubber (ethylene 20% and propylene 80%) and can be present in an amount by weight of the composition of 60%. Suitable products for the external layer are sold by Mitsubishi Chemical Company under the trade name Zelas 7023. Zelas 7023 is the subject of U.S. Patent Application Publication No. 2001/0034416 A1 which is incorporated herein by reference in its entirety and made a part hereof.

The intermediate layer can be a polymer blend of Zelas 7023 70% by weight and 30% by weight of a random copolymer of styrene and butadiene that has been hydrogenated. Suitable random copolymers of styrene and butadiene are sold by JSR under the trade name Dynaron 2320 P.

The external layer can be a polymer blend of 60% by weight Zelas 7023 and 40% by weight of a random copolymer of propylene and ethylene such as the copolymer sold under the trade name Novatec EG 7C. The films can display bi-modal behavior with peelable seals being formed at sealing temperatures of about 125° C. and permanent seals are obtained at about 160° C.

Other suitable films for this application include those disclosed in U.S. Pat. Nos. 5,849,843; 5,998,019; 6,083,587; 6,297,046; 5,139,831; 5,577,369; and U.S. Application No. 2003/0077466 A1, which are incorporated herein in their entirety by reference and made a part hereof.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A flexible container for an incompressible fluid, comprising a first chamber and a second chamber, said second chamber having therein a tube for providing fluid communication from the second chamber to the exterior of the container, the first chamber and the second chamber separated by a first peelable seal having a channel so constructed and arranged to allow a controlled quantity of said fluid to pass therethrough.

2. The container of claim 1, wherein the incompressible fluid comprises a liquid.

3. The container of claim 1, wherein the incompressible fluid comprises a flowable solid.

4. The container of claim 1, wherein the first peelable seal surrounds an opening of the tube attached to the container.

5. The container of claim 1, wherein the channel has a width greater than 5 mm.

6. The container of claim 1, wherein the channel has a width greater than 6 mm.

7. The container of claim 1, wherein the channel has a width greater than 7 mm.

8. The container of claim 1, wherein the container is made from a film comprising at least one material selected from the group consisting of polyolefins, polyamides, polyesters, polybutadiene, styrene and hydrocarbon copolymers, polyimides, polyester-polyethers, polyamide-polyethers, and combinations thereof.

9. The container of claim 8, wherein the film comprises at least one material selected from the group consisting of polyethylene homopolymers, ethylene α-olefin copolymers, polyethylene copolymers, polypropylene homopolymers, polypropylene copolymers, styrene and hydrocarbon random copolymers, styrene and hydrocarbon block copolymers, and combinations thereof.

10. The container of claim 1, wherein the first chamber is divided into a plurality of subchambers by one or more openable seals.

11. The container of claim 10, wherein the one or more openable seals comprise at least a second peelable seal.

12. The container of claim 11, wherein the first peelable seal and the second peelable seal can be activated by a force ranging from about 3 N/15 mm to about 30 N/15 mm.

13. The container of claim 11, wherein the first peelable seal has a first activating force and the second peelable seal has a second activating force, the second peelable seal activating force is greater than the first peelable seal activating force.

14. The container of claim 13, wherein the difference between the first peelable seal activating force and the second peelable seal activating force is greater than about 1 N/15 mm and less than about 10 N/15 mm.

15. The container of claim 1, wherein the first peelable seal is openable in response to fluid pressure applied to the first peelable seal.

16. The container of claim 11, wherein the second peelable seal is openable in response to fluid pressure applied to the second peelable seal.

17. The container of claim 1, wherein at least a portion of the first peelable seal comprises a shape selected from the group consisting of semicircular, rectangular, trapezoidal, polygonal, and combinations thereof.

18. The container of claim 1, wherein at least a portion of the channel comprises a shape selected from the group consisting of straight, rectangular, trapezoidal, polygonal, and combinations thereof.

19. A method of sterilizing a container, the method comprising:
providing a container comprising a first chamber comprising an incompressible fluid and a second chamber connected to a tube, the first chamber and the second chamber separated by a first peelable seal having a channel so constructed and arranged to allow a controlled quantity of the fluid to pass therethrough; and
sterilizing the container and allowing the fluid from the first chamber to flow through the channel into the second chamber to equalize the pressure in the first chamber and the second chamber.

20. The method of claim 19, wherein the container further comprises a third chamber separated from the first chamber by a second peelable seal.

21. The method of claim 19, wherein the sterilization is performed by heat sterilization.

22. The method of claim 19, wherein the first peelable seal surrounds the tube.

23. The method of claim 19, wherein the channel has a width greater than 5 mm.

24. The method of claim 19, wherein the channel has a width greater than 6 mm.

25. The method of claim 19, wherein the channel has a width greater than 7 mm.

26. The method of claim 19, wherein at least a portion of the first peelable seal comprises a shape selected from the group consisting of semicircular, rectangular, trapezoidal, polygonal, and combinations thereof.

27. The method of claim 19, wherein at least a portion of the channel comprises a shape selected from the group consisting of straight, rectangular, trapezoidal, polygonal, and combinations thereof.

28. The method of claim 19, wherein the incompressible fluid comprises a liquid.

29. The method of claim 19, wherein the incompressible fluid comprises a flowable solid.
